(19) United States
(12) Patent Application Publication
(10) Pub. No.: US 2017/0273602 A1
(43) Pub. Date: Sep. 28, 2017

(54) SYSTEM FOR DEFINING AND EXECUTING AUDIOMETRIC TESTS

(71) Applicant: AUDYX SYSTEMS LTD., Jerusalem (IL)

(72) Inventors: Yves Samuel LASRY, Nantes (FR); Philippe Joseph COHEN, Jerusalem (IL)

(21) Appl. No.: 15/503,725

(22) PCT Filed: Mar. 3, 2015
(86) PCT No.: PCT/IB2015/051542

§ 371 Date: Feb. 14, 2017

Related U.S. Application Data
(60) Provisional application No. 62/037,149, filed on Aug. 14, 2014.

(51) Int. Cl. A61B 5/12; A61B 5/00

(52) U.S. Cl. CPC A61B 5/123; A61B 5/0022; A61B 5/7435

(57) ABSTRACT

A hearing test system comprising: a computer system comprising a visual display for a user to select out of a plurality of sound types a desired sound type of a test sound for transmission to a subject and to select out of a plurality of computer-based input events a desired computer-based input event for registering a subject response to said test sound.

### RELATED APPLICATIONS

This application claims benefit under 35 U.S.C. 119(e) of U.S. Provisional Application 62/037,149 filed Aug. 14, 2014, the disclosure of which is incorporated herein by reference.

### FIELD OF THE INVENTION

Embodiments of the invention relate to defining and executing an audiometric test.

### BACKGROUND

A hearing test, or alternatively an audiometric test, is a test for measuring hearing acuity, and is generally administered to a subject with an audiometer. The audiometer is configured to provide a set of sounds (“test sounds”) having well-defined parameters, which are presented to a subject as stimuli to which he or she is prompted to respond. The test sounds may be produced by, for example, an earphone, an earphone insert, bone-conduction vibrator, and/or an open-field speaker. Audiometric tests may measure one or more of various parameters relating to hearing acuity, including threshold of audibility (an intensity of a sound to just be audible to a subject) at each of a plurality of frequencies in a range of frequencies, ability to distinguish between different sound intensities or different sound frequencies, recognize speech or distinguish speech from background noise. A test sound may, by way of example, comprise a pure tone characterized by a particular frequency and a particular intensity. In another example, the test sound may comprise a complex sound that is a combination of two or more pure tones. A complex sound may be characterized by a substantially continuous frequency spectrum. The complex sound may optionally be a natural sound or a vocal sound.

An audiometric test procedure typically includes a series of test sound presentations followed by registering a subject's response to the presented test sounds. The response of the subject to the test sound is recorded and analyzed to produce an assessment of the subject's hearing. The subject response may be, by way of example, the subject's acknowledgement that the test sound was perceived, the subject's indication that an audible sound is no longer heard, or the subject's assessment of whether the test sound produced discomfort or not. Alternatively or additionally, the subject response may also include by way of example a correct identification by the subject of one or more aspects of the presented test sound. For example, the subject may be presented with a vocal test sound that is a spoken word, sentence or logatom, which may be referred to generically herein as “word”. The subject may respond by correctly repeating the presented word or selecting the correct word from a selection provided to the subject on a computer screen. Alternatively or additionally, the subject may be presented with two similar test sounds and be asked to characterize a difference between the test sounds including by way of example, higher or lower in pitch, different word, louder or softer in intensity, different in apparent position of sound source.

### SUMMARY

An aspect of an embodiment of the invention relates to providing a system, hereinafter also referred to as “Audyx”, that enables an audiologist to relatively easily define and/or use any one or more of a variety of hearing tests for testing a subject's hearing.

Audyx optionally comprises a computer system (hereinafter “the Audyx computer system”) that includes or has access to an instruction set stored in a non-transitory computer readable medium, which the Audyx computer system executes to generate a visual display (“test composing display”) on a display screen. An audiologist may manipulate the test composing display to define a hearing test having a desired test sound or sequence of test sounds for transmitting to a subject and optionally having a desired input event for registering subject responses to the test sound. For convenience of presentation, a hearing test defined using Audyx may be referred to as an “Audyx hearing test”, and an audiologist that defines a new Audyx hearing test using Audyx may be referred to herein as a “test composer”.

Optionally, Audyx comprises an acoustic system for transmitting test sounds to a subject and a test response system for registering a subject's response to the test sounds. A controller responsive to an Audyx hearing test controls the acoustic system to transmit to the subject a sequence of test sounds in accordance with the Audyx hearing test and controls the test response system to register subject responses to the test sounds in accordance with the Audyx hearing test. The acoustic system may comprise at least one or any combination of more than one of an earphone, an earphone insert, bone-conduction vibrator, and/or an open-field speaker. The test response system may comprise at least one or any combination of computer input devices such as a microphone, a keyboard, mouse, a touch screen, and/or a gesture recognition system.

Various types of test sounds may be used to test hearing acuity of subjects. In accordance with an embodiment of the invention, test sounds may be grouped in a defined set of categories of sound types (hereinafter “sound categories” or “SoundCats”) available for selection by a test composer to define a given Audyx hearing test. One example of a SoundCat is a pure tone, characterized by a single tone frequency. Another example of a SoundCat is a warble tone characterized by having a tone frequency that changes over the duration of the tone. Other examples of SoundCats include noise and vocalized words. In an embodiment of the invention, the test composing display provides a plurality of visual representations of SoundCats that may be manipulated by the test composer to select SoundCats for inclusion in the Audyx hearing test. The test composer may define, for each selected SoundCat, a set of variables (“output variables”) that further characterize the test sounds. Examples of output variables include intensity, duration, and number and frequency of repetitions.

The test composing display may enable a test composer to select multiple SoundCats to provide a “compound test sound” comprising a mixture of sounds from different SoundCats. For example, a test composer may select, in the test comprising display, a word SoundCat and a noise SoundCat in order to provide a compound test sound having a vocalized word transmitted to the subject together with noise. Such a compound test sound may be used to determine a subject's ability to distinguish vocalized words against a background of noise.

Optionally, upon selecting a plurality of SoundCat test sounds to provide a compound test sound, the test composing display may present to the test composer output variables particular to the compound test sound. For example, to define the word/noise compound test sound, Audyx may provide the test composer with a volume ratio output variable for defining a ratio, or range of ratios, between a volume of the vocalized word and a volume of the background noise. Optionally, values for output variables are set to change over time or repetitions.

In an embodiment of the invention, the test composing display may provide the test composer with visual representations of “input options”, each input option corresponding to an “input event” for registering and/or analyzing responses of the subject to a given test sound. The test composer may manipulate one or more visual representations of input formats presented on the test composition display to select the desired input event format(s).

A given input event may be characterized as being in at least one of the following input event categories: a subject-generated registration event, in which the subject interacts with a test registration system to register a response to a test sound; an observer-generated registration event, in which an audiologist observing the subject interacts with a test registration system to register the subject's response to a test sound; and an automated registration event in which the test response system passively monitors the subject and automatically detects and registers subject responses. An example of a subject-generated registration event includes the subject entering through a test response system an answer to a question regarding a test sound. An example of an observer-generated registration event includes an audiologist observing the subject and entering an answer to a question regarding the subject's response to a test sound. An example of an automated registration event includes a test response system that comprises a camera monitoring the subject and automatically detecting and registering body movements by the subject during and/or after presentations of a test sound. Optionally, each presentation of a test sound is coupled with or followed by an input event, so that each test sound presentation is associated with a registration of the subject's response.

Optionally, the registered subject's responses are collected and analyzed and/or stored for subsequent analysis. The analysis may include any of various graphical and/or tabular forms and using any of various statistical criteria for categorizing the responses.

In an embodiment of the invention, test sounds in an Audyx hearing test may be responsive to subject responses to test sounds from a previously executed Audyx hearing test. For example, an incorrect subject response to a vocal test sound at a given volume may result in vocal test sounds being transmitted at an increased volume in a subsequent execution of the Audyx hearing test.

In an embodiment of the invention, Audyx acquires a collection of a plurality of registered subject responses from a plurality of different Audyx hearing tests conducted on a population of patients. The collection of the plurality registered subject responses may provide a database of subject responses. In an embodiment of the invention, the database may be analyzed to provide statistics for use in analyzing and diagnosing responses of a given subject to a given test sound and comparing the given subject's individual responses to responses averaged over a population of subjects.

In an embodiment of the invention, Audyx acquires a plurality of Audyx hearing tests defined by any one of a plurality of test composers to provide a database of Audyx hearing tests for selection and use in conducting a hearing test.

The Audyx computer system may optionally be centralized within a single computer device, or be a “distributed system” with code and hardware components located in different locations. In an embodiment of the invention, the Audyx computer system may comprise a local computer device located at an audiometry test site together with the headphones and/or a remote computer system with which the local computer device communicates. The remote computer system may optionally be a remote server or a remote distributed system. The remote distributed system may be referred to herein as a “cloud computer system.”

In the discussion, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical features that appear in more than one figure are generally labeled with a same label in all the figures in which they appear. A label labeling an icon representing a given feature of an embodiment of the invention in a figure may be used to reference the given feature. Dimensions of features shown in the figures are chosen for convenience and clarity of presentation and are not necessarily shown to scale.

FIGS. 1A-1D shows schematic illustrations of an Audyx system in accordance with an embodiment of the invention;

FIGS. 2-13 schematically show an exemplary sequence of SoundCat and SoundCat variable selections via a test composing display in accordance with an embodiment of the invention;

FIG. 14 schematically shows a representation of a series of test sounds produced in an Audyx hearing test in accordance with an embodiment of the invention;

FIG. 15 schematically shows an exemplary test response system in accordance with an embodiment of the invention;

FIG. 16 schematically shows an analysis of registered subject responses gathered through an execution of an Audyx hearing test in accordance with an embodiment of the invention;

FIG. 17 schematically shows an alternative analysis of registered subject responses gathered through an execution of an Audyx hearing test in accordance with an embodiment of the invention.

FIGS. 18-19 schematically show an exemplary sequence of SoundCat and SoundCat variable selections via a test composing display in accordance with an embodiment of the invention; and

FIG. 20 schematically shows a representation of a series of test sounds produced in an Audyx hearing test in accordance with an embodiment of the invention.

### DETAILED DESCRIPTION

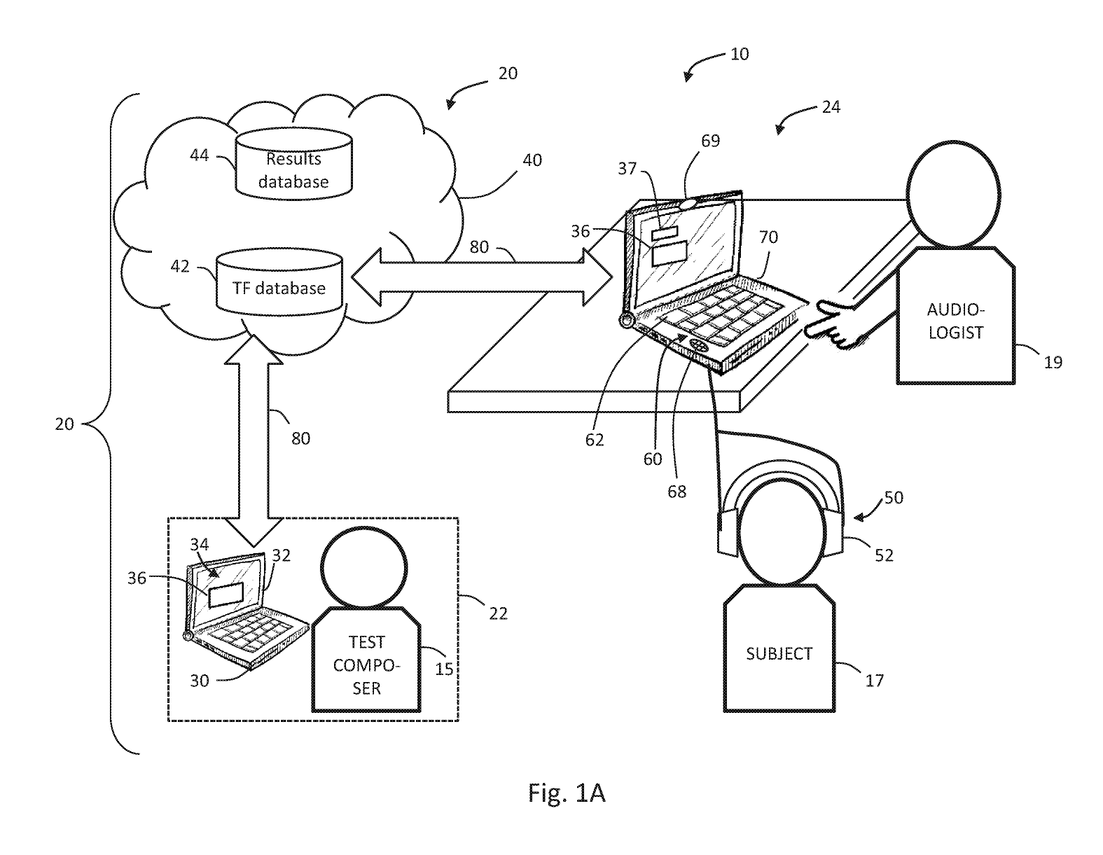

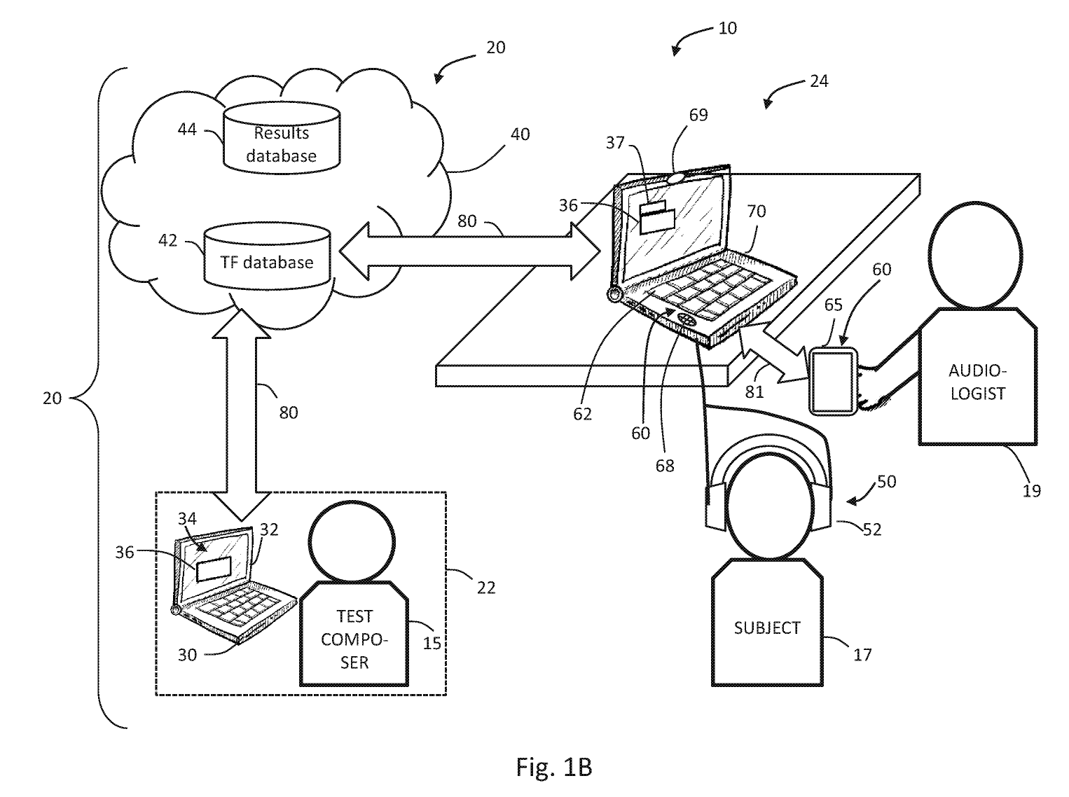

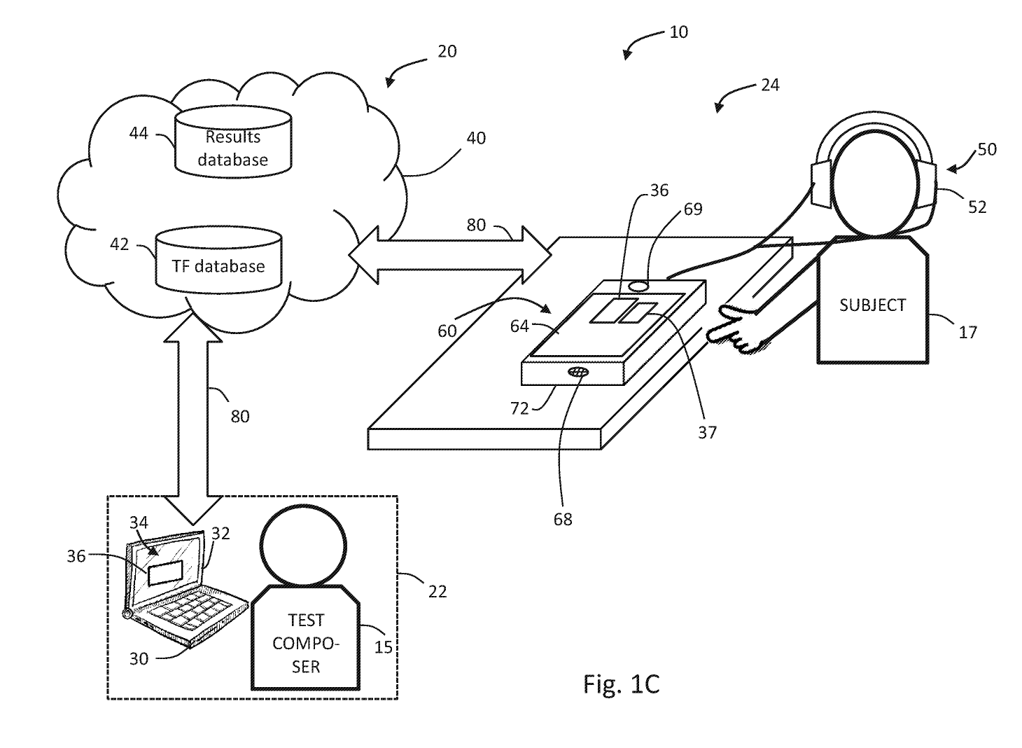

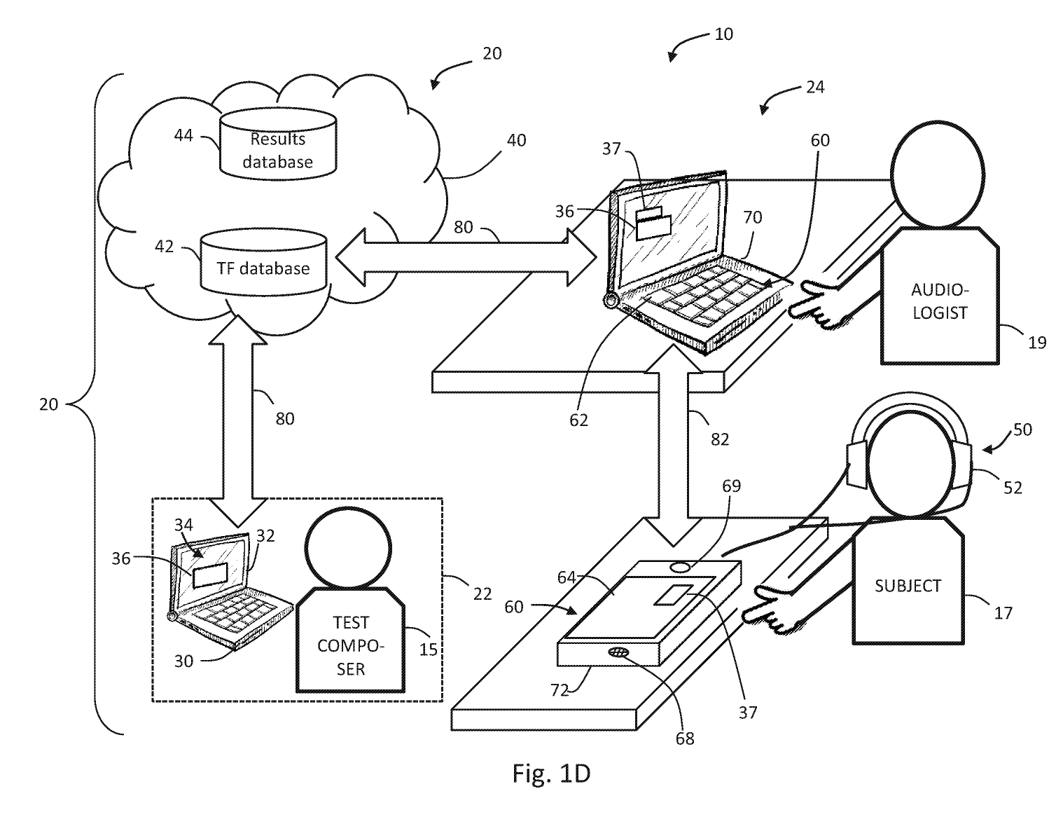

FIGS. 1A-1C schematically shows a Audyx system 10 in accordance with an embodiment of the invention. Audyx comprises an Audyx computer system 20 that includes or has access to an instruction set stored in a non-transitory computer readable medium, which the Audyx computer system executes to generate a visual display 34 (“test composing display”) that a test composer may manipulate to compose an Audyx hearing test to define a desired arrangement of test sounds to be presented to a subject, as well as optionally a desired arrangement of subject responses to test sounds.

In an embodiment of the invention, Audyx computer system 20 may comprise a computer device 30 having a display screen 32 on which a test composing display 34 may optionally be displayed. Computer device 30 may be operated by test composer 15 who manipulates test composing display 34 to create a new Audyx hearing test.

Test composing display 34 may create a test file, schematically represented by a block 36 that encodes a given Audyx hearing test created by test composer 15 manipulating test composing display 34. A given test file 36 may be transferred to other computer systems or devices (for example, a computer device 70 connected to an appropriate communication channel schematically represented by double headed block arrow 80), as described further hereinbelow) to recreate the encoded Audyx hearing test at a later time and/or in a different computer system or device.

Optionally, computer device 30 with which test composer 15 composes an Audyx hearing test may optionally be located in a first location that is schematically represented by dashed box 22, and acoustic system 50 and a test response system 60 with which an Audyx hearing test is executed may be located in a second location 24 that is relatively remote from first location 22.

Audyx computer system 20 may optionally comprise a cloud computer system 40, may comprise a database 42 (“test file database” or “TF database”) comprising a plurality of test files 36 stored in a non-transitory computer readable medium, each test file representing a given Audyx hearing test. Optionally, cloud computer system 40 and computer device 30 may communicate with each other via an appropriate communication channel 80. By way of example, test file 36 stored in TF database 42 may be sent to or accessed by test composing display 34 displayed in computer device 30, so that, through interacting with test composing display 34, test composer 15 may view and/or edit an Audyx hearing test represented by test file 36. Alternatively or additionally, test file 36 created through test composer's 15 manipulation of test composing display 24 may be stored TF database 42 in cloud computer system 40.

In an embodiment of the invention, test file 36 stored in TF database 42 may, via appropriate communication channel 80, be sent to or accessed by an acoustic system 50 and/or a test response system 60 to execute a given Audyx hearing test encoded in the test file.

In an embodiment of the invention, acoustic system 50 may execute an Audyx hearing test, transmitting to a subject 17 an arrangement of test sounds encoded in a given test file 36, which is optionally downloaded or accessed from TF database 42. Acoustic system 50 may comprise at least one or any combination of more than one of an earphone (as schematically shown in FIGS. 1A-1C), an earphone insert (not shown), bone-conduction vibrator (not shown), and/or an open-field speaker (not shown). The open field speaker may be a plurality of open field speakers. The plurality of open field speakers may be a multichannel audio system, for example a 5.1 surround sound system or a 7.1 surround sound system. By way of example, acoustic system 50 as schematically shown in FIG. 1A comprises earphones 52 worn by a subject 17.

In an embodiment of the invention, test response system 60 may execute an Audyx hearing test, registering the subject's response to test sounds in accordance with test file 36. Test response system 60 may comprise at least one or any combination of computer input devices such as a keyboard 62 (as schematically shown in FIGS. 1A, 1B and 1D), a mouse (not shown), a touch pad (not shown) a touch screen 64 (as schematically shown in FIGS. 1C and 1D), a microphone 68 (as schematically shown in FIGS. 1A-1D), and/or a video camera 69 (as schematically shown in FIGS. 1A-1D) that is optionally operatively connected to a gesture recognition system (not shown).

Optionally, test response registrations made through test response system 60 during a given Audyx hearing test may be recorded to generate a file (a “subject response file”; not shown) stored in a non-transitory computer readable medium encoding a record of the subject's responses to test sounds as registered through test response system 60. Optionally Audyx computer system 20, optionally remote computer system 40, comprises a database 44 (“results database”) comprising a plurality of subject response files registered from one or more particular executed hearing tests.

Acoustic system 50 and test response system 60 may be controlled by a controller, schematically represented by a block 37 that is responsive to test file 36. Controller 37 may be an instruction set stored in a non-transitory computer readable medium and executed by a microprocessor housed with or operatively connected to acoustic system 50 and/or test response system 60, by way of example in computer device 70 and/or a computer device 72.

It will be appreciated that Audyx 10 in accordance with an embodiment of the invention is not necessarily limited to particular arrangements or locations of test composing display 34, acoustic system 50 and test response system 60 in particular computer devices.

Optionally, as schematically shown in FIGS. 1A-1C, acoustic system 50 and test response system 60, are housed in and/or operatively connected to a same computer device (for example, computer device 70 in FIGS. 1A-1B and computer device 72 in FIG. 1C). Alternatively, acoustic system 50 and test response system 60 may be housed in separate computer devices, for example, computer devices 70 and 72 as shown in FIG. 1D. The computer device in which acoustic system 50 and/or test response system 60 are housed may comprise, by way of example, a desktop computer, laptop computer, or a portable computer device such as a tablet computer, a handheld computer or a smartphone.

With reference to FIG. 1B, by way of example, test response system 60 may be fully or partially housed in an auxiliary computing device 65 (hereinafter an “ACD”) that is operatively connected to computer device 70 through an appropriate communication channel schematically represented by double headed block arrow 81. ACD 65 may be used by audiologist 19 as an alternative method of registering responses of subject 17 to test sounds. The ACD may comprise a portable computer device such as a tablet computer, a handheld computer or a smartphone. By way of example, audiologist 19 may register responses of subject 17 to test sounds through a touchscreen 67 housed in ACD 65. The use of portable ACD 65 provides improved freedom of motion for the audiologist to better observe the subject when registering subject response. Alternatively, audiologist 19 may be located remotely with respect to subject 17 and communicating with and/or observing subject 17 through one or more electronic communication means, by way of example, intercom, telephone, video conference, or instant messaging (“chat”).

By way of example, test composing display 34 may be provided in computer device 70 operated by audiologist 19, so that the same audiologist who composes an Audyx hearing test as a test composer also executes the hearing test (not shown).

By way of another example, as schematically shown in FIG. 1C, acoustic system 50 and test response system 60 (by way of example comprising touch screen 64) may be housed in or operatively connected to computer device 72 operated by subject 17. Optionally, subject 17 engages in a self-administered Audyx hearing test, in which subject 17 controls the presentation of test sounds and registers his or her own responses to test sounds through test response system 50, without or with minimal interaction with an audiologist during execution of the Audyx hearing test.

By way of another example, as schematically shown in FIG. 1D, test response system 60 may be housed in two computer devices, a first computer device 70 operated by audiologist 19 and a second computer device 72 operated by subject 17, in which audiologist 19 controls the presentation of test sounds and subject 17 registers his or her own responses to the presented test sounds. Acoustic system 50 may be operatively connected to computer device 72, as shown in FIG. 1D, or with computer device 70. Computer device 70 and computer device 72 may communicate with each other via an appropriate communication channel, schematically represented by double headed block arrow 82.

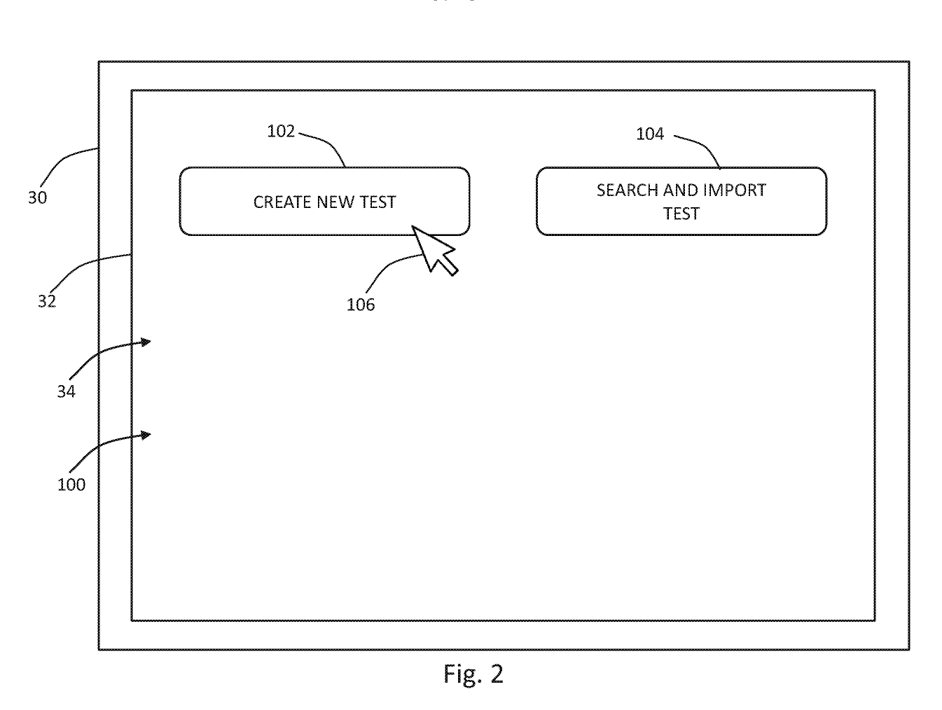

FIGS. 2-12 schematically show an exemplary sequence of SoundCat and SoundCat variable selections made, by way of example, by test composer 15 composing an Audyx hearing test via manipulating test composing display 34 shown in display screen 32 of computer device 30 (as schematically shown in FIGS. 1A-1D).

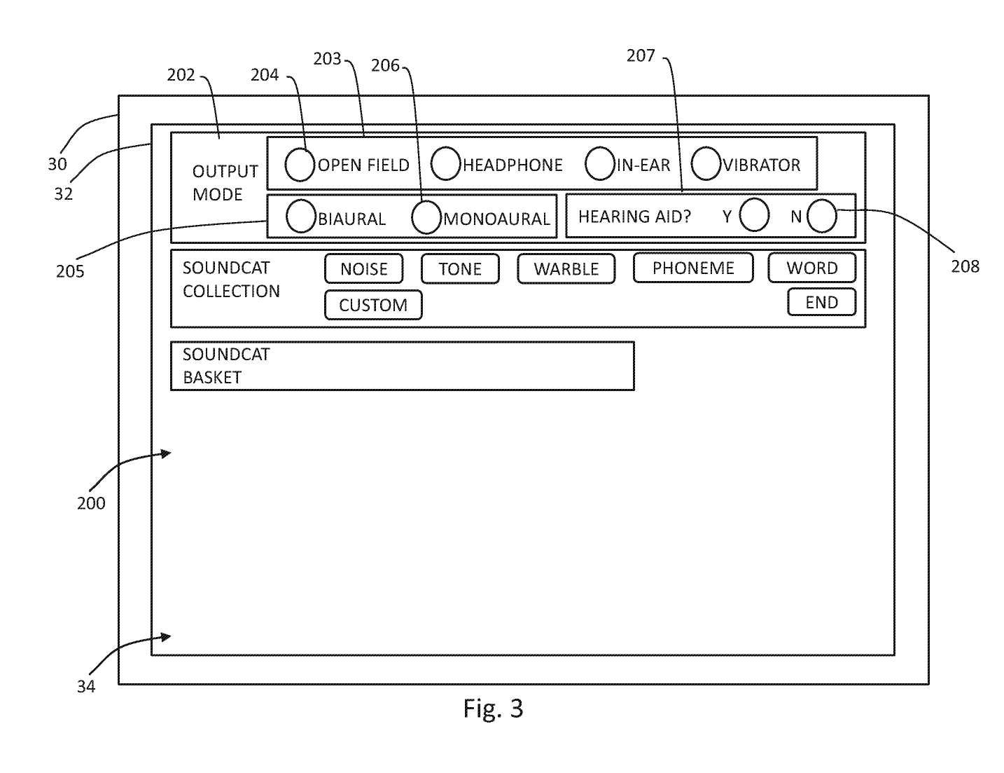

FIG. 2 schematically shows a starting page 100 of test composing display 34 showing display screen 32 of computer device 30 operated by a test composer (by way of example test composer 15 as schematically shown in FIGS. 1A-1D) composing a new Audyx hearing test. In accordance with an embodiment of the invention, the Audyx hearing test may define a sequence of test sounds to be presented to a subject (by way of example subject 17 shown in FIGS. 1A-1D) by an acoustic system (for example acoustic system 50 shown in FIGS. 1A-1D) during the execution of the Audyx hearing text, as well as a sequence of computer input events for registering the subject's response through a test response system (for example test response system 60 shown in FIGS. 1A-1D. Test composer 15 is prompted with two choices, to begin creating a new test through selecting icon 102, for example by clicking on the icon with a mouse, as indicated by the placement of mouse pointer icon 106. Selection of icon 102 leads to a test composing page 200 in a default state (as schematically shown in FIG. 3), which the test composer may manipulate to define a new Audyx hearing test. Selection of “search and import test” icon 104 leads to test composing display 34 showing a menu (not shown) of available “published” Audyx hearing tests, optionally from TF database 42 (as schematically shown for example in FIG. 1A). The selection of a published Audyx hearing test leads to a completed test composing page 200 (completed) that is populated with hearing test definitions in accordance with a test file corresponding to the selected Audyx hearing test. The test composer may manipulate the populated test composing page to optionally modify, and/or execute the selected test.

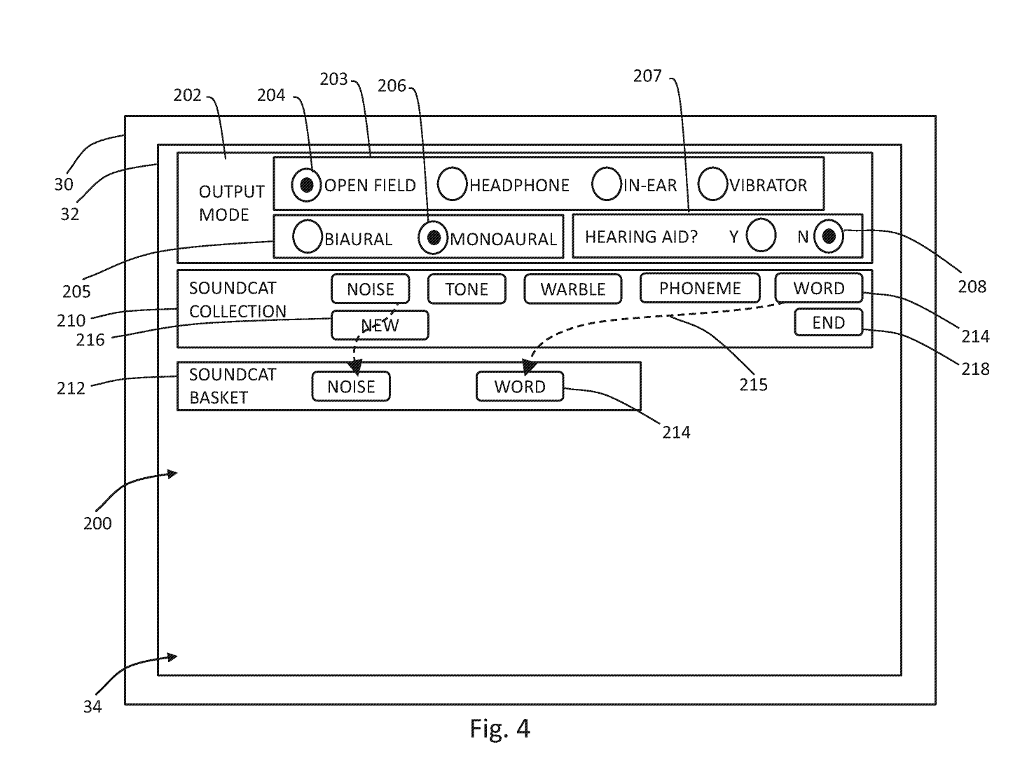

Reference is now made to FIG. 3, which schematically shows a test composition page 200 in a default state, which a test composer manipulates to define an Audyx hearing test, as schematically shown in FIG. 4.

Test composition page 200 may comprise by way of example an output mode selector 202 for the test composer to define various modes of test sound delivery. Selecting particular test sound delivery modes may, for example, define properties of signals encoding test sounds that are optimized for the particular modes of test sound delivery. By way of example, output mode selector 202 comprises a transducer selector 203 having radio buttons 204. By operating radio buttons 204, the test composer may select the type of transducer used for producing test sounds defined in the Audyx hearing test, for example, open field speakers, headphones, in-ear inserts and bone-conducting vibrators. Output mode selector 202 may further, by way of example, comprise binaural/monoaural selector 205 having radio buttons 206 for the test composer to define test sounds as being binaural or monoural, as well as by way of example hearing aid mode selector 207 having radio buttons 208.

By way of example, a SoundCat collection 210 as schematically shown in FIGS. 3-4 includes, by way of example, a plurality of SoundCat icons 214. In an embodiment of the invention, selection of a given SoundCat icon into a SoundCat basket 212 defines a given category of sound that will be presented as test sounds in the Audyx hearing test. As schematically shown in FIG. 4, the test composer may select a SoundCat in test composing display 34 by manipulating a visual representation of a desired SoundCat, by way of example dragging a desired SoundCat icon 214 from SoundCat collection 210 to a SoundCat basket 212. The manipulation of the desired SoundCat icon for selection may additionally or alternatively comprise clicking or double clicking on the desired SoundCat icon with the mouse, or clicking on a checkbox next to the desired SoundCat icon. If test composing display 34 is displayed on a touch screen, the manipulation of the desired SoundCat icon for selection may additionally or alternatively comprise tapping or double tapping a region of a touchscreen (not shown) occupied by the desired SoundCat icon, or tapping on a checkbox next to the desired SoundCat icon.

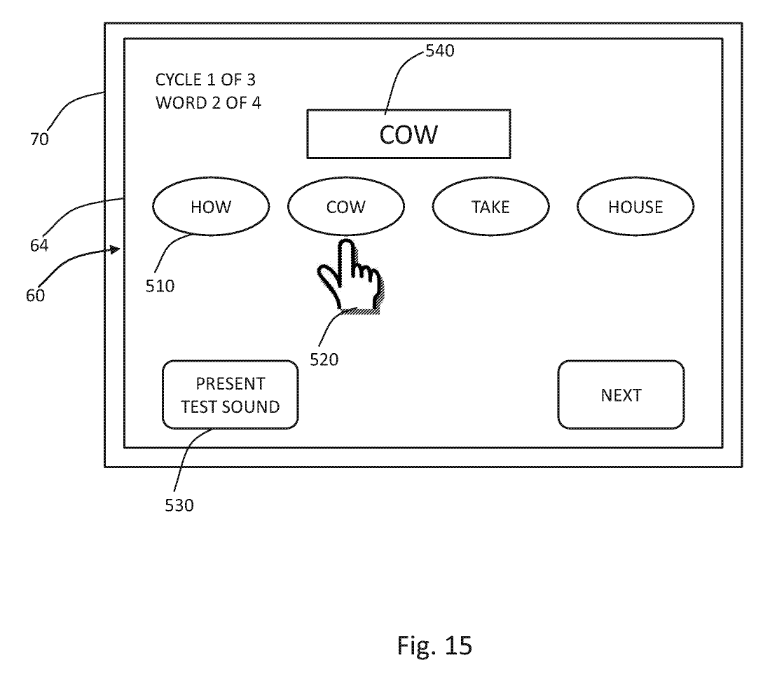

The test composer may select additional SoundCats by dragging additional desired SoundCat icons 214. The selection of multiple SoundCats may result in a compound test sound comprising multiple test sounds presented in a temporally overlapping manner. By way of example, the test composer drags two SoundCat icons 214, schematically represented by two broken arrows 215, the first SoundCat icon containing the term “word” and the second SoundCat icon containing the term “noise”, from SoundCat collection 210 to SoundCat basket 212. A hearing test defined by the selection of “word” and “noise” SoundCats may produce a compound test sound that presents a word test sound combined with a noise test sound (see for example FIG. 15). Upon completion of the dragging, copies of the dragged SoundCat icons 214 appear within SoundCat basket 212, indicating successful selection.

SoundCat collection 210 may optionally include a SoundCat icon 216 containing the term “new”, for creating a new SoundCat.

After the desired SoundCats are selected, the test composer may confirm the SoundCat selection and proceed with the test file composition by clicking on icon 218 containing the term “end”.

Whereas in FIG. 4, the test composer has successfully selected desired SoundCats 214, he has not as yet in FIG. 4 defined SoundCat variables for each of the selected Soundcats, respectively, which further characterizes the test sounds, and the sequence of the test sounds, to be presented to the subject during execution of the Audyx hearing test.

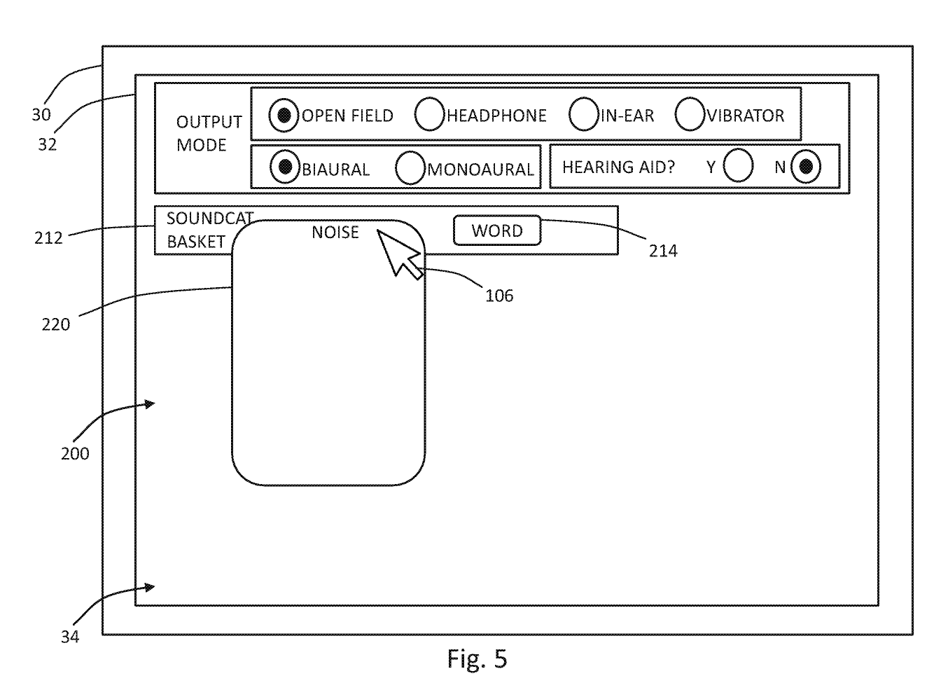

Reference is now made to FIG. 5. The audiometer may direct mouse pointer icon 106 and click on one of SoundCat icons 214 in SoundCat basket 212 to open an output variable definition window (OVD window) 220 for a given selected SoundCat. OVD window 220 shows a variety of output variables for the given SoundCat that is to be defined by the test composer. By way of example, in FIG. 5, the test composer directs mouse pointer icon 106 and clicks on the SoundCat icon containing the term “noise” to expand the icon into corresponding OVD window 220.

In an embodiment of the invention, the output variables that OVD window 220 prompts the test composer to define may be responsive to the particular corresponding SoundCat type. By way of example, a pure tone SoundCat may be defined by output variables such as, by way of example, pitch, volume and duration. A voice SoundCat may be defined by a different set of output variables including, by way of example, language, a selection of a pre-prepared word list, and volume.

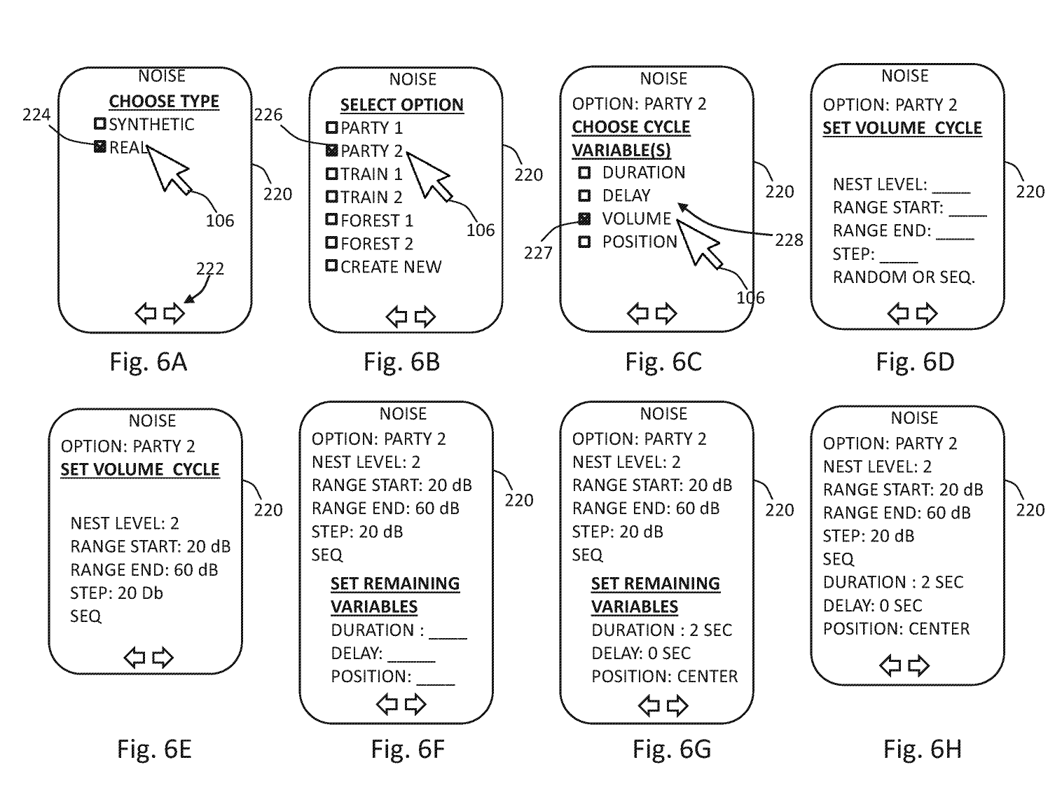

Reference is now made to FIGS. 6A-6H, which schematically shows manipulation of OVD window 220 by the test composer for defining output variables for the noise SoundCat. OVD window optionally guides the test composer through a series of queries to define output variables appropriate for the noise SoundCat. Optionally, OVD window 220 comprises arrow icons 222 for the test composer to navigate backward and forward through the sequence of queries presented in OVD window 220.

As schematically shown in FIG. 6A, upon opening OVD window 220, OVD window 220 presents the test composer by way of example, with a menu of basic noise types 224 to select from, “synthetic” and “real” noise, whereupon the test composer selects real noise. Synthetic noise may comprise, by way of example, white noise, brown noise, pink noise, blue noise, violet noise, or grey noise. Real noise may comprise a recording or simulation of a noisy environment, by way of example, a train station, a busy street, a party, a forest, rainfall, or the like.

As schematically shown in FIG. 6B, the test composer's selection of real noise triggers OVD window 220 to present a menu of real noise examples 226, including by way of example “party 1”, “party 2”, “train 1”, “train 2”, “forest 1” and “forest 2” for the test composer to select from, whereupon the test composer selects the desired real noise example 226.

As schematically shown in FIGS. 6C-6H, upon the test composer selecting the desired real noise example 226, he is presented with a selectable list of output variables controlling how the particular noise example 226 will be presented in the Audyx hearing test. By way of example, output variables characterizing a noise SoundCat may include “duration” that defines the duration of each instance of test sound, “delay” that defines a delay in test sound initiation in relation to other SoundCats (if any), “volume” that defines a volume of the test sound and “position” that defines an apparent spatial position of the source of the test sound.

Typically, an Audyx hearing test comprises a series (“test sound series”) of multiple test sound presentations, with each test sound presentation followed by a registering of a subject's response to the test sound. The test sound series or a portion thereof may be characterized with a “cycle” of test sounds in which each of a plurality of test sounds presents, respectively, a range of a given output variable. The hearing test may be defined to include a plurality of nested cycles so that more than one output variable range is tested over the course of the hearing test.

As schematically shown in FIG. 6C, the test composer selects from a selectable list 228 which output variable(s) will characterize a cycle. By way of example, the test composer selects the desired “volume” variable 227 so that the hearing test will include a volume cycle presenting the party 2 noise at a range of volumes.

As schematically shown in FIG. 6D, OVD window 220 presents entry fields for output variables defining the volume cycle, including by way of example “nest level” defining how the volume cycle for the party 2 noise test sound interacts with other cycles, the starting volume for the cycle (“range start”), the ending volume for the cycle (“range end”) and the volume steps (“step”) between the starting volume and the ending volume. The test composer may optionally select if the range of volumes will be presented in sequence from the start volume to the end volume or if the range of volumes will be presented randomly (“random or seq.”). In an embodiment of the invention, the volume steps may be responsive to the subject's response to one or more earlier presented test sounds. By way of example, the volume step in the “step” field may be set as “reactive”, in which case the volume of the noise increases or decreases depending on the subject's response to the previous test sound.

As schematically shown in FIG. 6E, by way of example, the test composer enters a nest level of 2, a starting volume of 20 dB (decibel), an ending volume of 60 dB, and a volume step of 20 dB. The test composer also selects for the volume cycle to be presented sequentially. As a result, the hearing test will include a cycle (or cycles) of the party 2 noise being presented sequentially at three different volumes, at 20 dB, at 40 dB, and then at 60 dB.

As schematically shown in FIGS. 6F-6H, upon the test composer defining the output variables for the volume cycle, OVD window 220 prompts the test composer to enter values for the remaining output variables that were not selected to characterize a cycle. By way of example, the test composer enters a duration of 2 seconds, a delay of 0 second, and a position of center, so that the party 2 noise will be consistently presented during the hearing test with a duration of 2 seconds, a delay of 0 seconds in relation to other SoundCats, and with the apparent spatial position being center. Optionally, duration of a noise SoundCat may be defined as “continuous”, in which case a noise test sound corresponding to the selected SoundCat will be continuously presented to a subject during execution of the hearing test.

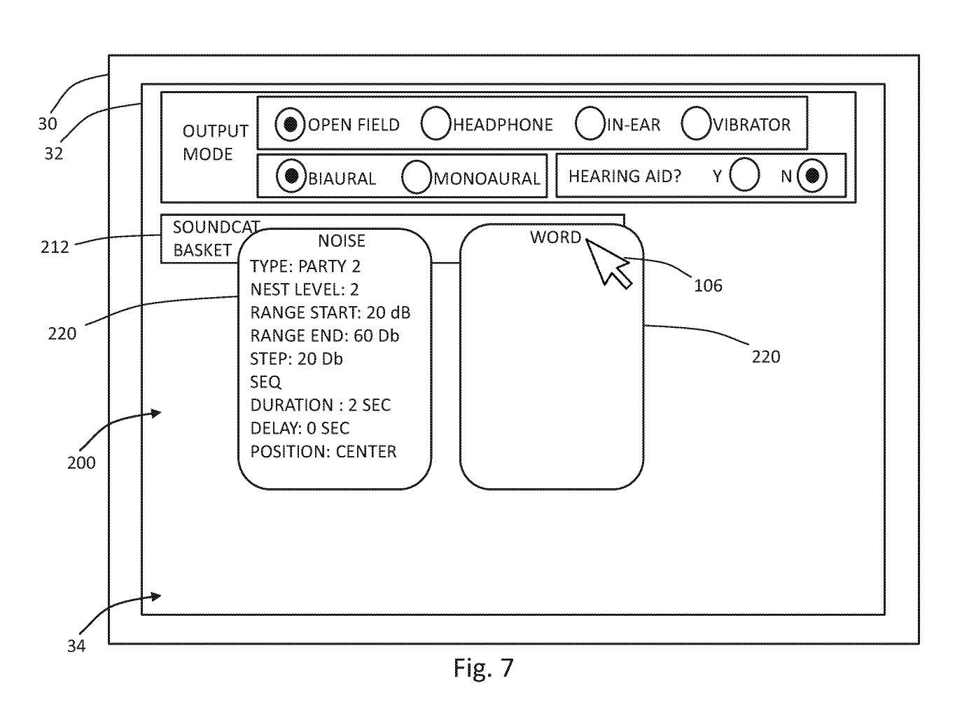

As schematically shown in FIG. 7, the test composer directs mouse pointer icon 106 and clicks on the SoundCat icon containing the term “word” to expand the icon into corresponding OVD window 230 to define output variables for the word SoundCat.

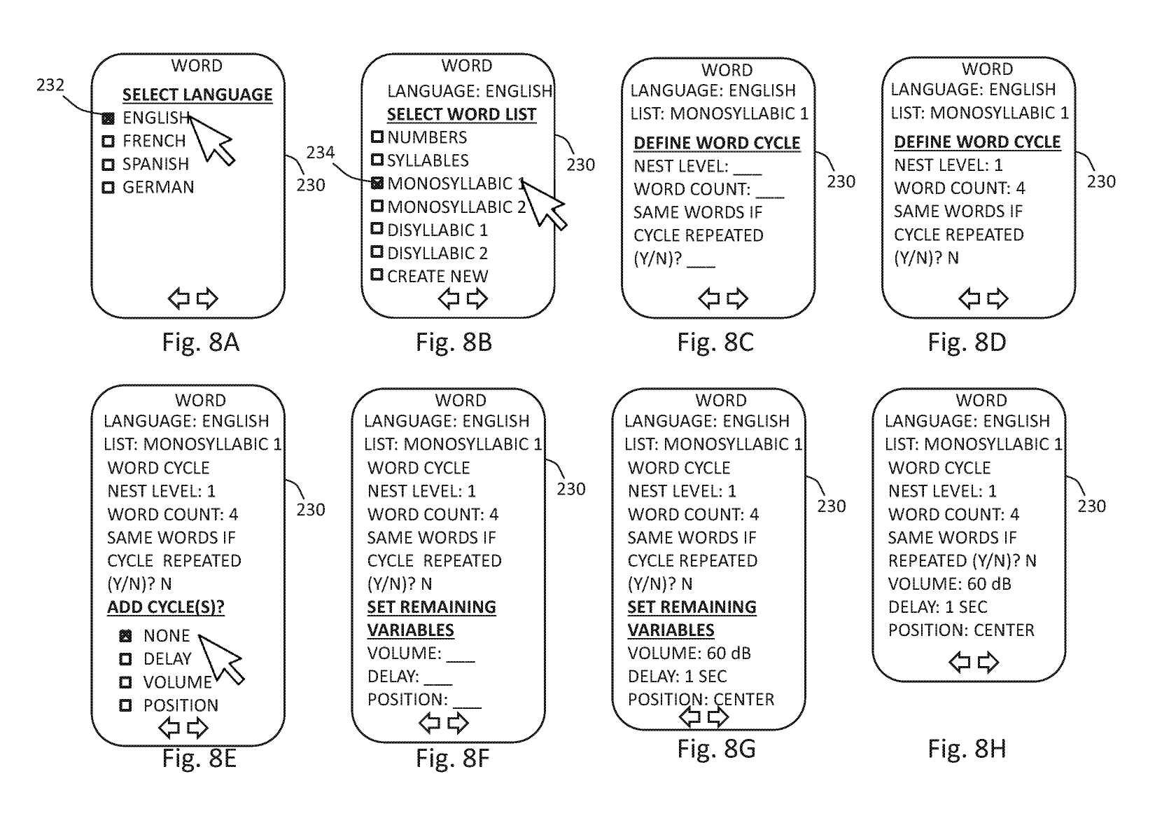

Reference is now made to FIGS. 8A-8H, which schematically shows manipulation of OVD window 230 by the test composer for defining output variables for the “word” SoundCat. The OVD window optionally guides the test composer through a series of queries to define output variables appropriate for the word SoundCat.

As schematically shown in FIG. 8A, upon opening OVD window 230 for the word SoundCat, OVD window 230 presents the test composer, by way of example, with a selectable list of languages 222 to select from, whereupon the test composer selects “English”.

As schematically shown in FIG. 8B, the test composer's selection of English triggers OVD window 230 to present a selectable list of English word lists 234, including by way of example numbers, monosyllabic words, and disyllabic words for the test composer to select from. By way of example, the test composer selects desired wordlist “monosyllabic 1”.

As schematically shown in FIG. 8C, OVD window 230 presents entry fields for output variables defining a word cycle, including by way of example “nest level” defining how the word cycle interacts with other cycles, the number of words presented in the word cycle (“word count”), and whether or not the same words are used if the word cycle is repeated (“same words if cycle repeated (y/n)?”).

By way of example, as schematically shown in FIG. 8D, the test composer enters a nest level of 1 and a word count of 4, and indicates that the word cycle does not repeat the same words if the cycle is repeated. As a result, the hearing test will include a cycle (or cycles) of 4 words being presented from the English monosyllabic 1 wordlist.

As schematically shown in FIG. 8E, upon the test composer completing the definition of the word cycle, OVD window 230 prompts the test composer to select an output variable, by way of example “delay”, “volume” or “position”, for creating another cycle if desired. By way of example, the test composer selects “none” to indicate that no further cycles are desired for the word Soundcat.

As schematically shown in FIGS. 8F-8H, upon the test composer indicating that no further cycles are desires in the word SoundCat, OVD window 230 prompts the test composer to enter values for the remaining output variables that were not selected to characterize a cycle. By way of example, the test composer enters a volume of 60 dB, a delay of 1 second, and a position of center, so that the word test sounds will be consistently presented in the hearing test with a volume of 60 dB, a delay of 1 second in relation to the noise SoundCats (having been defined with a delay of 0 seconds), and with the apparent spatial position being center.

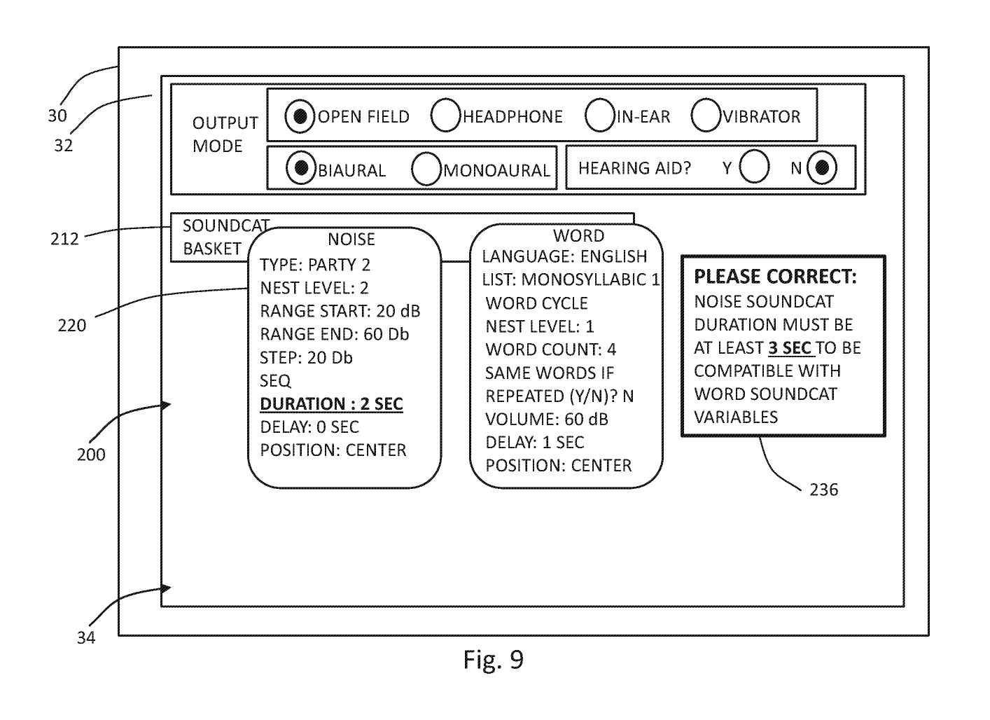

In an embodiment of the invention, test composing page 200 may check for incompatibilities among the selected SoundCats and their respective output variables defined by the test composer, and alert the test composer of incompatibilities if present. By way of example, as schematically shown in FIG. 9, test composing page 200 detects an incompatibility between the respective output variables defined in the noise SoundCat and the word SoundCat, where the 2 second duration of the noise SoundCat is too short to be presented together with the word SoundCat as defined. Optionally, test composing page 200 displays a warning message 236 describing the nature of the incompatibility. Optionally, composing page 200 will prevent the audiometer from proceeding further in the Audyx hearing test composition until the incompatibility is corrected.

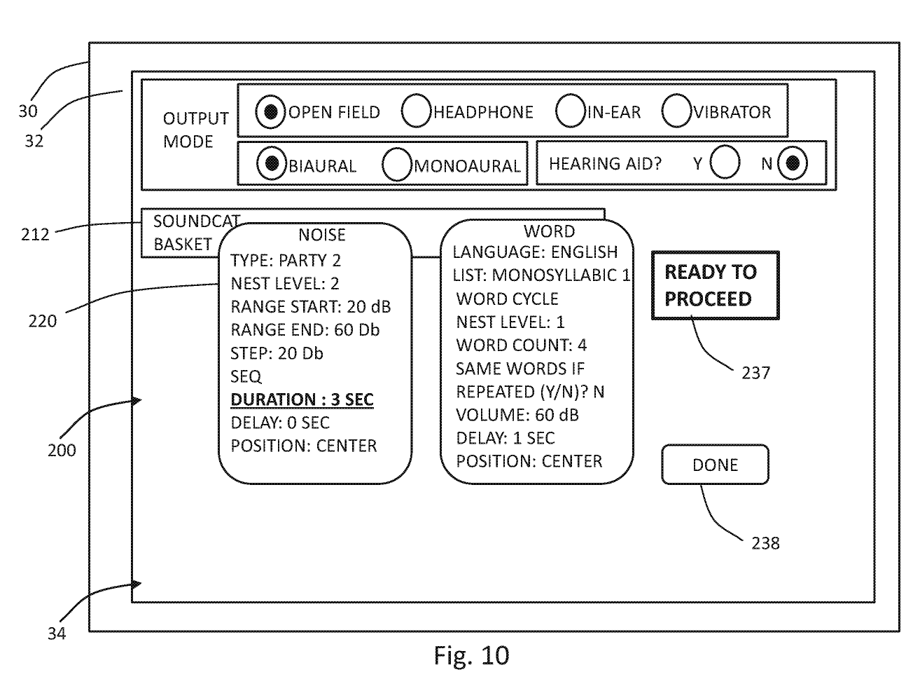

In an embodiment of the invention, the test composer may select the OVD window of the desired SoundCat to edit the output variables as needed. As schematically shown in FIG. 10, the test composer edits the noise SoundCat output variables to define the duration as 3 seconds to make the duration long enough to be compatible with the word SoundCat. As a result of the edited noise SoundCat duration, composing page 200 displays a confirmation message 237 comprising the phrase “Ready to Proceed”, and further displays a button 238 comprising the term “done” that the test composer may click on to proceed with the composition of the hearing test.

Whereas in FIGS. 5-10, the test composer has successfully defined output variables for the selected noise and word SoundCats, he has not as yet in FIG. 10 selected at least one input option, which defines an input event for registering via test response system 60 a subject's responses to test sounds.

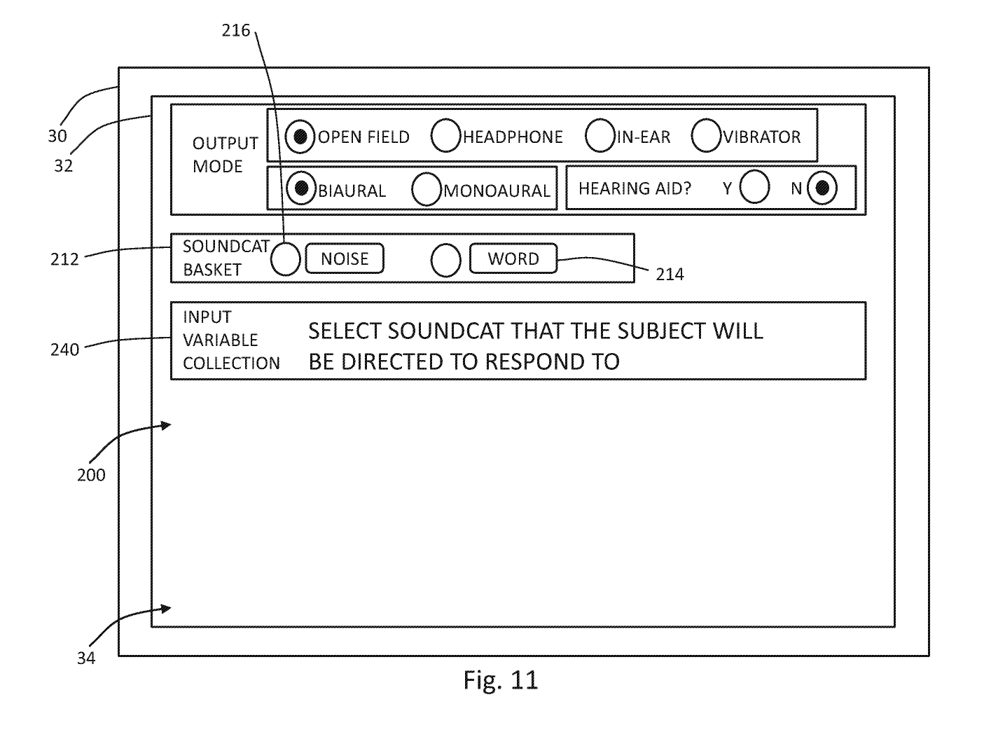

Reference is now made to FIG. 11. In an embodiment of the invention, input options available for selection may be responsive to one or more of SoundCats selected by the test composer. Upon the test composer successfully defining the output variables for the respective SoundCats, test composing page 200 optionally prompts the test composer to select, from the previously selected SoundCats placed in SoundCat basket 212, a desired SoundCat for defining a pool of input options available for selection. By way of example, test composing page 200 provides radio buttons 216 corresponding to respective Soundcat icons 214 to indicate the desired SoundCat for input option definition. By way of example, test composing page 200 includes an input option collection 240, which, prior to a selection of a desired input option definition, includes a message stating “select SoundCat that the subject will be directed to respond to”.

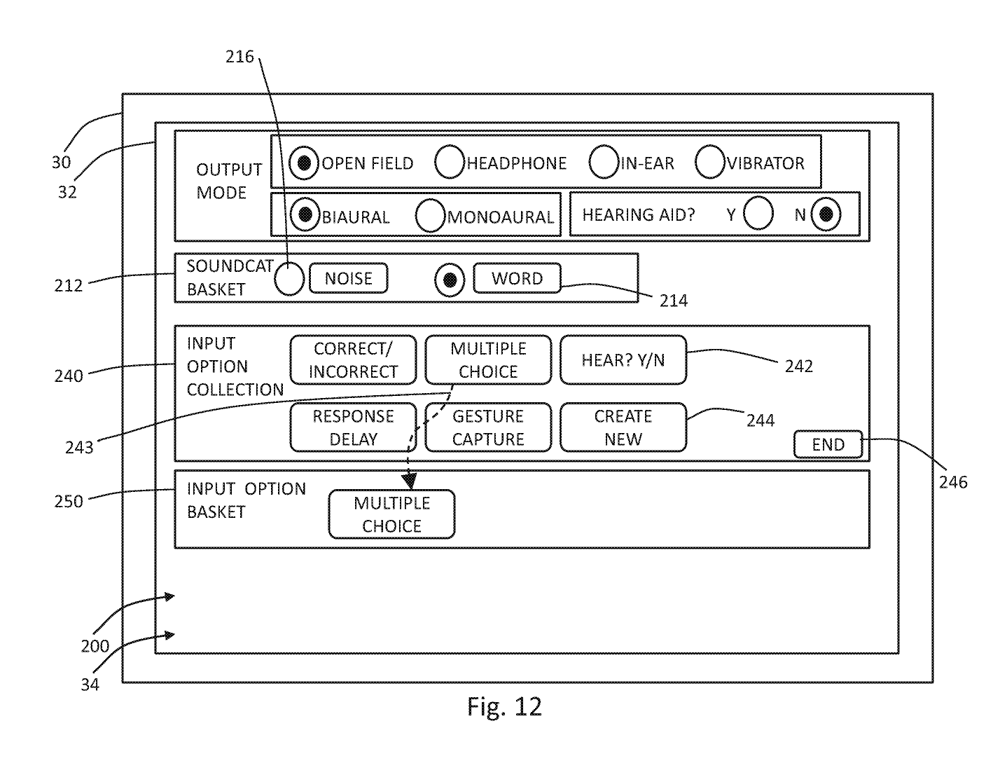

Reference is now made to FIG. 12. Upon the test composer selecting the desired SoundCat, input option collection 240 is populated with visual representations of input options, schematically represented as input icons 242 corresponding to input options available for selection. The selected input option defines a procedure by which an audiologist and/or a subject interacts with a test response system (such as test response system 60 shown in FIGS. 1A-1D) in accordance with an embodiment of the invention to register subject responses to test sound presentations produced by an acoustic system (such as acoustic system 50 shown in FIGS. 1A-1D) during the execution of an Audyx hearing test. A given input event may be characterized as being in at least one of the following input event categories: a subject-generated registration event, in which subject 17 interacts with a test registration system (by way of example test registration system 60 as shown in FIGS. 1A-1D) to register a response to a test sound; an observer-generated registration event, in which an audiologist 19 observing subject 17 interacts with a test registration system to register the subject's response to a test sound; and an automated registration event in which a test response system passively monitors the subject 17 and automatically detects the subject's responses to a test sound. Optionally, the registering of the presented test sound during an input event comprises entering an answer of a question presented to the person operating the test response system.

By way of example, a given subject-generated registration event may be a subject entering through a test response system an answer to a question regarding a test sound. By way of example, a given observer-generated registration event may be an audiologist observing the subject entering an answer to a question regarding the subject's response to a test sound. By way of example, a given automated registration event may be a video camera monitoring the subject, the video camera being operatively connected to a test response system comprising a gesture recognition system that detects and registers body movements by the subject during and/or after presentations of a test sound.

By way of example, upon the test composer's selection of SoundCat icon 214 corresponding to the word SoundCat results in input option collection 240 being populated with input icons 242. As schematically shown in FIG. 12, input icons 242 correspond, respectively, to a “correct/incorrect” input option, a “multiple choice” input option, a “hear? Y/N” input option, a “response delay” input option and a “gesture capture” input option. The “correct/incorrect” input option provides an observer-generated registration event in which an audiologist observing a subject is presented with a reference response to a test sound via a computer-controlled display, observes the subject to determine whether or not the subject's response to the test sound matches the reference response, and interacts with the test response system (by way of example as schematically shown in FIG. 1A) to register the correctness of the subject's response. By way of example, the audiologist may observe the subject to register whether or not the subject correctly repeated a vocalized word presented as a test sound. The “multiple choice” input option provides for a subject-generated registration event in which subject is presented, optionally via a computer-controlled display, with a choice of possible correct responses to the test sound, and interacts with the test response system to register what the subject considers to be the best choice out of the possible correct responses. The “hear? Y/N” input option provides for a subject-generated or observer-generated registration event in which a subject, or an audiologist observing the subject, interacts with the test response system to indicate if a given test sound was perceived by the subject. The “response delay” input option, which may be combined with a subject-generated response event, provides for an automated registration event, in which duration of time between a presentation of a given test sound and a response by the subject is registered with the subject-generated response event. The “gesture capture” input option provides for an automated registration event in which a test response system comprising a gesture recognition system coupled to a video camera that monitors the subject detects hand, body and/or facial gestures of the subject being presented with the test sound for automatically detecting subject responses to the test sound. The gesture capture input option is optionally selected together with at least one subject-generated event or observer-generated registration event. Other input options for hearing test subject response procedures may be provided in the input option collection. Other examples of input events defined by selecting input options include a procedure by which a subject indicates the apparent position of a source of a test sound, a procedure by which a subject indicates whether a test sound intensity is comfortable or uncomfortably loud.

By way of example, upon the test composer's selection of SoundCat icon 214 corresponding to the word SoundCat results in input option collection 240 being populated with input icons 242 respectively corresponding to a “correct/incorrect” input option, a “multiple choice” input option, a “hear? Y/N” input option, a “response delay” input option and a “gesture capture” input option. The test composer may select a desired input option by manipulating the visual representation of the desired input option, by way of example by dragging an input icon 242 corresponding to the desired input option into input option basket 250 as schematically indicated by broken arrow 243. By way of example, the test composer selects input icon 242 corresponding to the “multiple choice” input option. Optionally, the test composer may select more than one input option.

Input option collection 240 may optionally include an input option icon 244 containing the term “create new”, for creating a new input option.

After desired input options are selected, the test composer may confirm the input option selection and proceed with the test file composition by clicking on icon 246 containing the term “end”.

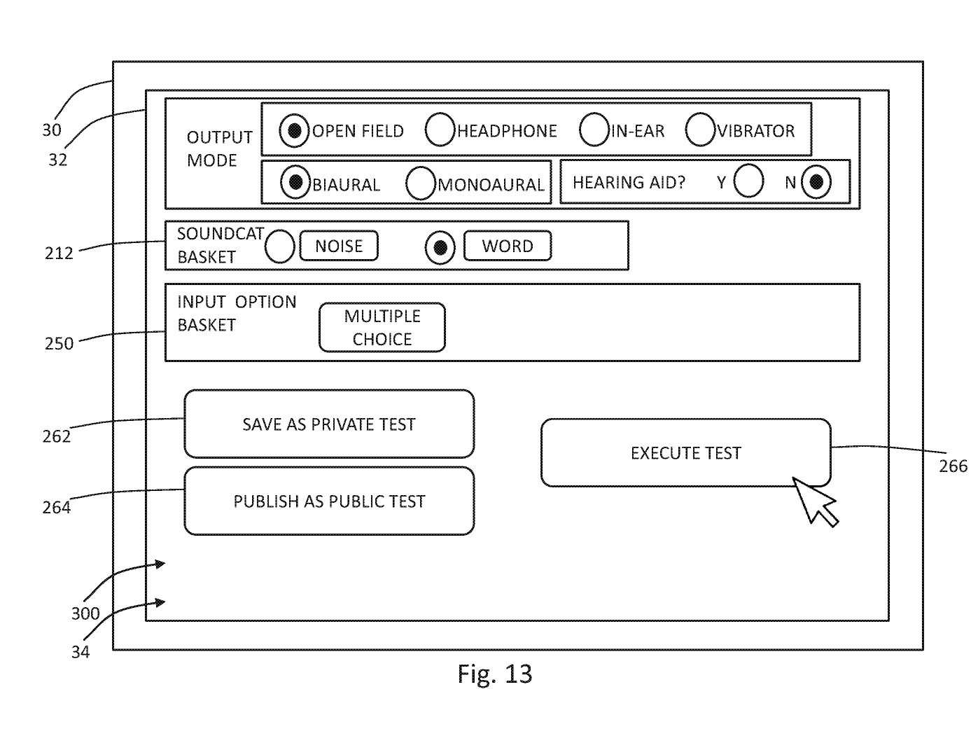

Reference is now made to FIG. 13, which schematically shows a final page 300 of test composing display 34, optionally having three buttons for the audiologist to select from. First button 262, consisting of the phrase “save as private test”, when selected, creates a new test file that is saved at a location selected by the audiologist. Second button 264, consisting of the phrase “publish as public test”, creates a new test file that is saved in TF database 42 in cloud computer system 40 (as schematically shown for example in FIGS. 1A-1C), so that it is accessible to other audiologists, optionally having sufficient authorization and optionally via the use of Audyx. Third button 266, consisting of the phrase “execute test”, instructs an appropriate acoustic system (such as acoustic system 50 shown in FIGS. 1A-1C) and an appropriate test response system (such as test response system 60 shown in FIGS. 1A-1C) to execute a hearing test in accordance with a test file corresponding to the SoundCats and variables defined in test composing display 34.

Optionally, the audiologist can manipulate test composing display 34 to change previously-made definitions of the Audyx hearing test prior to selecting any one of buttons 262, 264 and 266.

Optionally, upon the audiologist, at opening page 100 (see FIG. 2) of test composing display selecting button 104 consisting of the phrase “search and import test”, and selecting a desired test file, test composing display 34 may proceed to final page 300 having definitions populated in accordance with the selected test file. The audiologist may execute the Audyx hearing test as defined, or change one or more populated definitions as desired and then execute the hearing test.

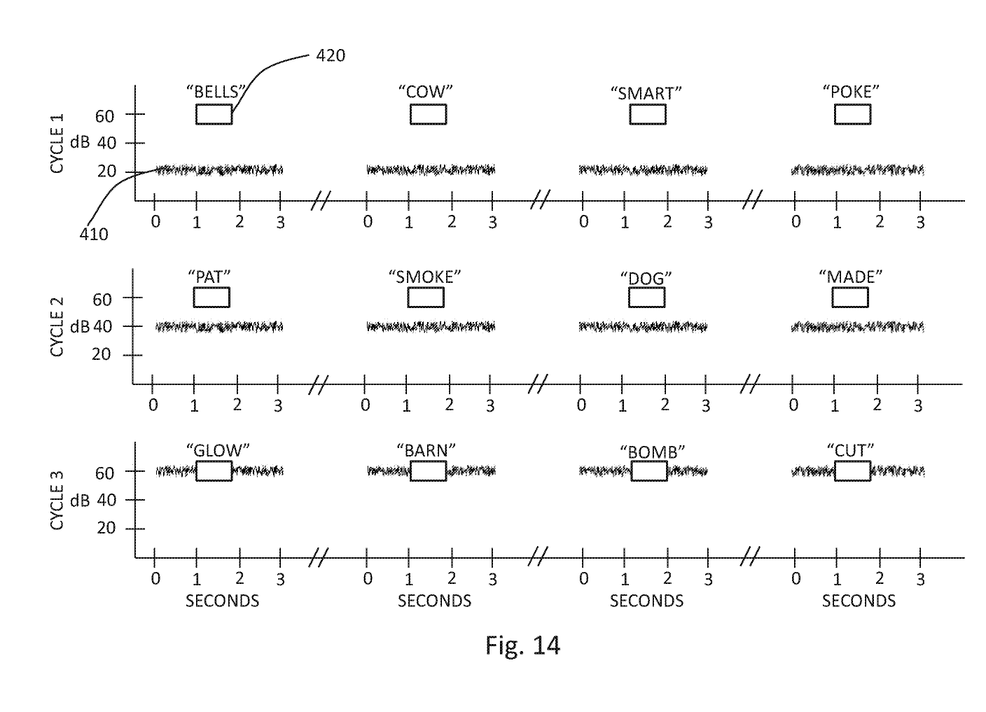

Reference is now made to FIG. 14, which schematically shows test sounds presented by an acoustic system in accordance with an embodiment of the invention executing an Audyx hearing test as defined in FIGS. 2-13. The Audyx hearing test includes cycles 1 through 3, and each cycle respectively includes the presentation of four compound test sounds comprising a noise test sound defined by a noise SoundCat and a word test sound defined by a word SoundCat. Cycle 1 presents four compound test sounds: three seconds of the party 2 noise 410 presented at 20 dB combined with the word “bells” 420 presented at 60 dB starting 1 second after the start of the party 2 noise; three seconds of the party 2 noise 410 presented at 20 dB combined with the word “cow” 420 presented at 60 dB starting 1 second after the start of the party 2 noise; three seconds of the party 2 noise 410 presented at 20 dB combined with the word “smart” 420 presented at 60 dB starting 1 second after the start of the party 2 noise; and three seconds of the party 2 noise 410 presented at 20 dB combined with the word “poke” 420 presented at 60 dB starting 1 second after the start of the party 2 noise. Cycles 2 and 3 repeat a cycle of four word test sounds in a similar fashion, but with different words presented each time and with the accompanying party 2 noise being presented at 40 dB in cycle 2 and at 60 dB at cycle 3.

As schematically shown for example in FIG. 6H, the hearing test presents a cycle of the noise test sound being presented at 20 dB, 40 dB and 60 dB at a nest level of 2. As schematically shown for example in FIG. 8H, the hearing test present a cycle of the word test sound having a word count of four words at a nest level of 1, with no word repetitions if the word cycle is repeated. The nest level defines the hierarchy of the cycles, such that the cycle with a lower nest level is completed before the cycle that with a higher nest level. Therefore, as schematically shown in FIG. 14, the four word presentation of the word cycle, having a lower next level, is repeated within each 20 dB step of the noise cycle.

Reference is now made to FIG. 15, which schematically shows an exemplary test response system 60 in accordance with an embodiment of the invention executing a hearing test as defined in FIGS. 2-13, having the “multiple choice” input option as a selected input option. The test response system may be touchscreen 64 housed in computer device 70 as schematically shown in FIG. 1B. Touchscreen 64 shows a multiple choice procedure in execution, and includes four answer selection buttons 510. A subject interacting with touchscreen 64, represented by hand icon 520, presses on button 530 consisting of the phrase “present test sound” in order to present a test sound, then selects one of the answer selection buttons that the subject believes was the word presented in the test sound together with noise. The selected answer is presented in field 540 so that the subject can ascertain that the desired answer was selected. When the subject is ready to move on to the next test sound, the subject presses on the button consisting of the term “next”.

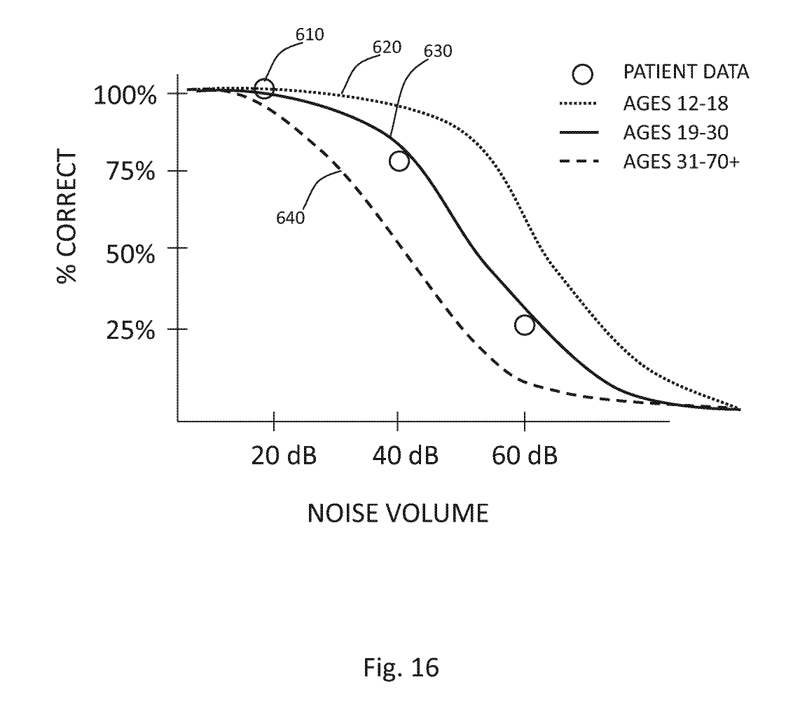

After an Audyx hearing test is performed, the subject responses may be analyzed to assess the hearing of the subject. The format of analysis of the subject responses may be defined as a variable within a selected input option (not shown). FIG. 16 schematically shows an analysis of registered subject responses gathered through an execution of an Audyx hearing test in accordance with an embodiment of the invention, by way of example an analysis of one or more repetitions of the hearing test schematically shown in FIG. 15. Circular plots 610 plots accuracy of the subject's perception of the presented words, presented as the percentage of correct answer selected (y axis) against the volume of noise presented together with the words as a compound test sound. By way of example, the subject's accuracy decreases with volume of accompanying noise, from about 100% accurate at 20 dB of noise, to about 75% accuracy at 40 dB of noise, to about 25% accuracy at 60 dB of noise. In an embodiment of the invention, subject's plot 610 may be compared to other results, optionally accessed from results database 44 for example as schematically shown in FIG. 1B. By way of example, subject's plot 610 may be compared to combined results of substantially the same hearing test conducted with other subjects, grouped in three age groups and shown as dotted line 620 showing the relationship between accuracy and noise volume in subjects of ages 12-18, solid line 630 showing the relationship between accuracy and noise volume in subjects of ages 19-30, and dashed line 640 showing the relationship between accuracy and noise volume in subjects of ages 31-70+.

In an embodiment of the invention, an Audyx hearing test may adjust output variables responsive to results of analysis and proceed to automatically execute again with the adjusted output variables. By way of example, if the subject of the result as schematically shown in FIG. 16 is sixteen years old, and therefore has a response accuracy that is lower than expected for his age cohort, Audyx may proceed to repeat the same Audyx hearing test but with the word SoundCat being defined at a higher volume (for example 70 dB instead of 60 dB) and/or with the noise SoundCat being defined at a lower range of volumes (for example 15 to 55 dB instead of 20 dB to 60 dB).

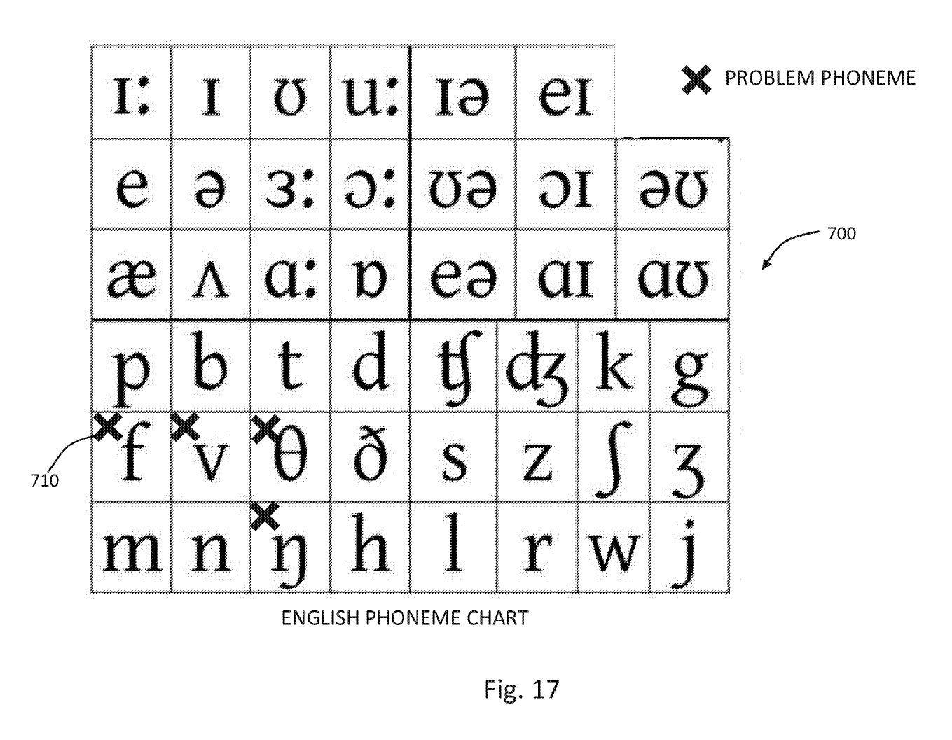

FIG. 17 schematically shows an alternative analysis of registered subject responses gathered through an execution of an Audyx hearing test in accordance with an embodiment of the invention, for example repetitions of the hearing test schematically shown in FIG. 15. Optionally, one or more of the answer selection buttons 510 (as schematically shown in FIG. 15) in a multiple choice subject response registration procedure may consist of a word or a logatom that differs with the word of the test sound in one phoneme (for example “cow” compared with “how”). The likelihood by which a mistaken response is based on a difference of a given phoneme may be tabulated over the course of repetitions of the hearing test schematically shown in FIG. 15. If a relatively high percentage of mistaken answers is correlated to particular switched phonemes, those particular phonemes may be distinguished as “problem” phonemes that the subject has trouble accurately perceiving. FIG. 17 shows a chart 700 of known phonemes in the English language. The problem phonemes may be marked with an x-shaped icon 710 on chart 700, so that an audiologist may easily identify phonemes that the subject has relative difficulty in perceiving.

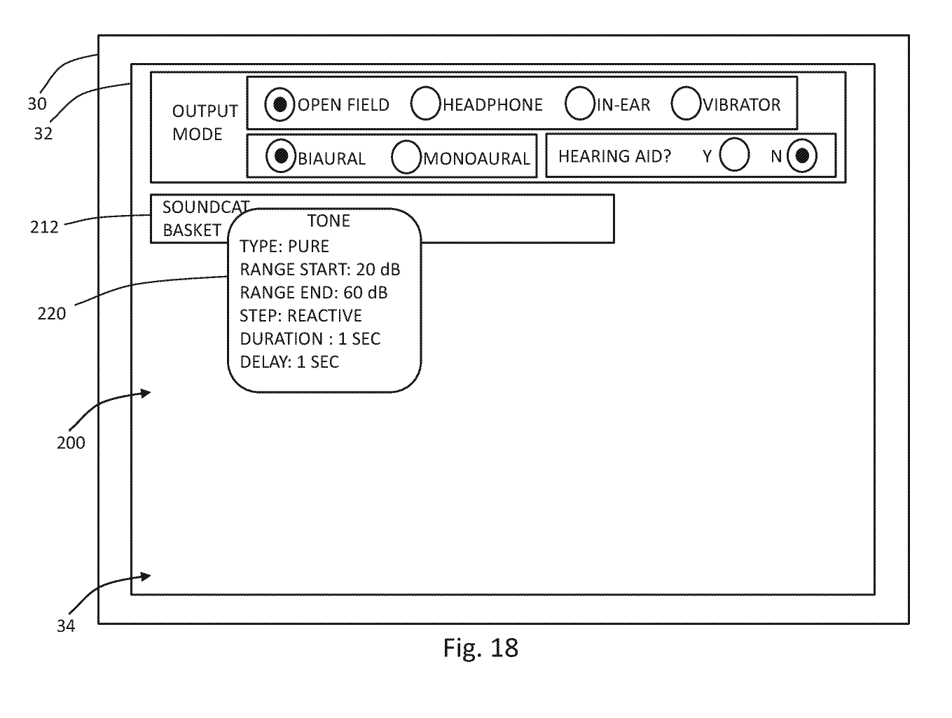

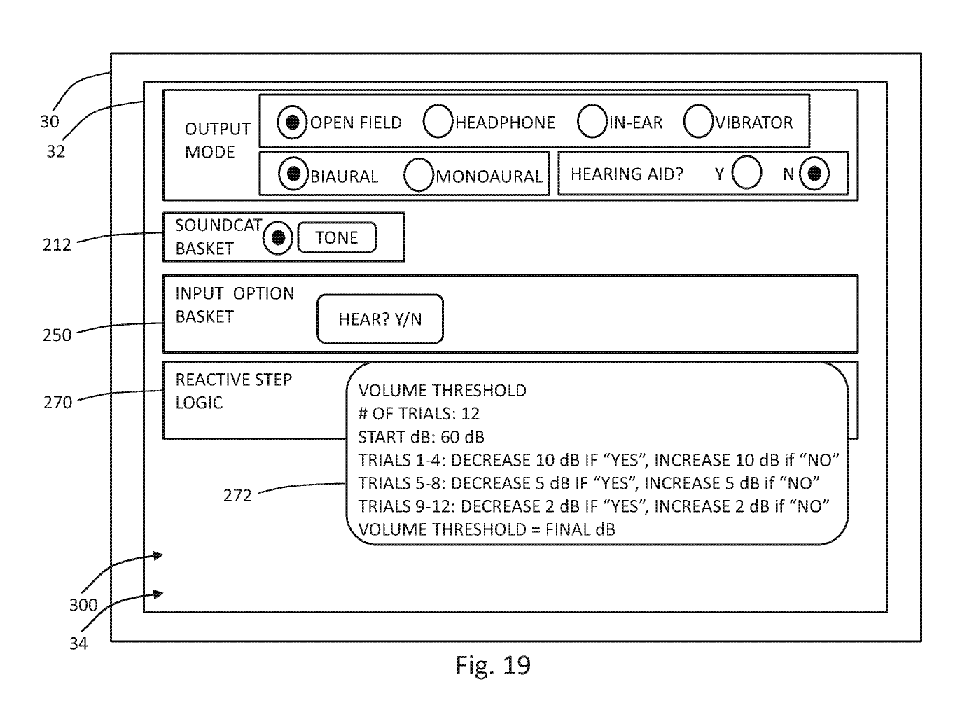

FIGS. 18-19 schematically show an exemplary sequence of SoundCat and SoundCat variable selections made, by way of example, by test composer 15 composing an Audyx hearing test via manipulating test composing display 34 shown in display screen 32 of computer device 30 (as schematically shown in FIGS. 1A-1D).

Test composing display 34 as shown in FIGS. 18 and 19 are substantially the same as the test composing display shown in FIG. 3-13, with the exception that the tone Soundcat was selected instead of the noise and tone Soundcats. As shown in FIG. 18, in the output variable definition window (OVD window) 220, the test composer defines test sound as being a pure tone ranging in intensity between 20 dB and 60 dB, with a duration of 3 seconds and a delay of 1 second for each test sound presentation (or “trial”). The step is defined as “Reactive”, meaning that a change in dB from trial to trial is responsive to the subject's response to the previously presented test sound.

As shown in FIG. 19, the test composer has previously dragged “Hear? Y/N” into an input option basket 250 to select the desired input option. Accordingly, when the given Audyx hearing test is executed, a subject will be presented with a test sound, then prompted to respond if the subject heard a sound or not. Because, as shown in FIG. 18, the “step” in OVD window 220 for the tone Soundcat was defined as “reactive”, the test composer is presented with a Reactive step logic field 270, in which the test composer defines a set of rules 272 by which the test sound intensity is responsive to the subject's previous response. By way of example, as shown in FIG. 19, rule set 272 is defined as a volume threshold test, where the subject is presented with 12 pure tone trials beginning with a pure tone of 60 dB. In trials 1-4, if the subject hears the test sound, the intensity of the next pure tone test sound is decreased by 10 dB, and if the subject does not hear the test sound, the intensity of the next pure tone test sound is increased by 10 dB. In trials 5-8, the intensity of the next pure tone test sound is decreased by 5 dB if the subject hears the test sound and increased by 5 dB if the subject does not hear the test sound. In trials 9-12, the intensity of the next pure tone test sound is decreased by 2.5 dB if the subject hears the test sound and increased by 2.5 dB if the subject does not hear the test sound.

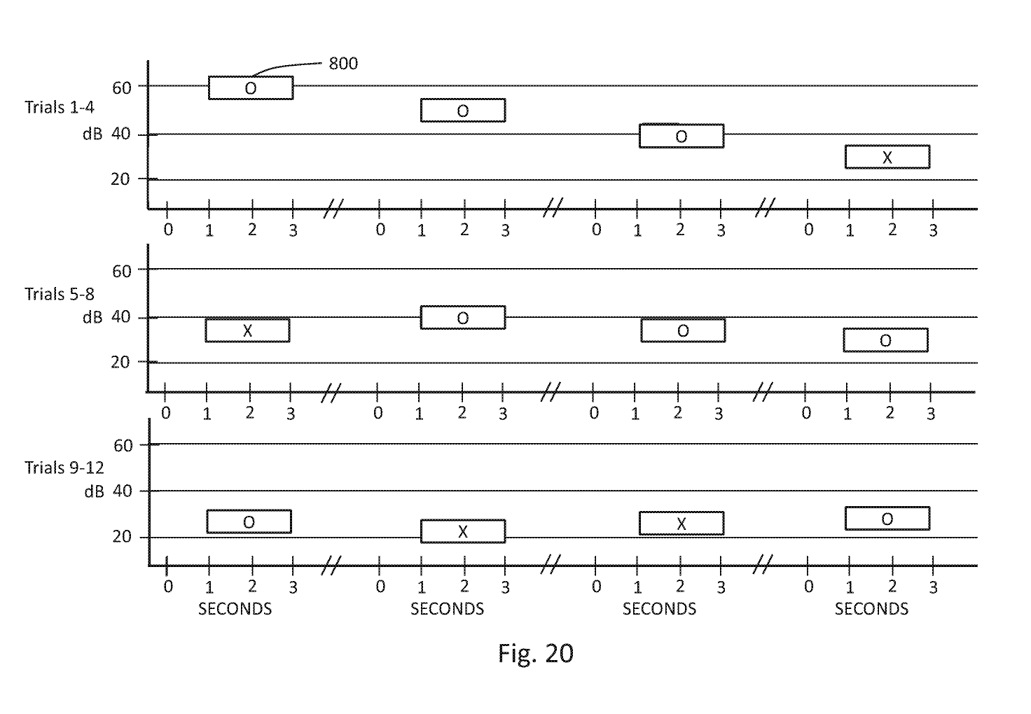

Reference is now made to FIG. 20, which schematically shows test sounds presented by an acoustic system in accordance with an embodiment of the invention executing an Audyx hearing test as defined in FIGS. 18 and 19. The Audyx hearing test includes trials 1 through 12, and each cycle respectively includes the presentation of a pure tone test sounds defined by a tone SoundCat. Each trial presents a pure tone 800. Pure tone 800 is presented at 60 dB in the first trial. In the subsequent trials, the intensity of the test sound is responsive to the subject's response to the previous test sound, as defined in rule set 272. Each test sound to which the subject responds as having heard is schematically marked with an “0” and each test sound to which the subject respond as having not heard is schematically marked with an “X”. In accordance with rule 272, each test sound marked with an “0” is followed by a new test sound that is lower in intensity and each test sound marked with an “X” is followed by a new test sound that is higher in intensity. In an embodiment of the invention, the degree to which the following test sound's intensity is responsive to the subject's response to the previous test sound decreases over the course of the trials, so that a volume threshold is reached.

Optionally, an Audyx hearing test may similarly test a comfort threshold of a subject, where the intensity of a later test sound increases if the subject responds that an earlier test sound was comfortable and the intensity of the following test sound increases if the subject responds that the earlier test sound was uncomfortably loud. Optionally, an Audyx hearing test may similarly test sound source location, where intensity of a tone presented in the left ear is increased (and/or the tone presented in the right ear is decreased) if the subject responds that a perceived source of the test sound is located to the right of the subject, and intensity of a tone presented in the left ear is decreased (and/or the tone presented in the right ear is increased) if the subject responds that a perceived source of the test sound is located to the right of the subject. Optionally, an Audyx hearing test may similarly test an intelligibility threshold of a subject, where the intensity of a vocalized word increases relative to a background noise (or the intensity of the background noise decreases relative to the vocalized word) if the subject fails to correctly identify an earlier presented vocalized word, and the intensity of the vocalized word decreases relative to the background noise (or the intensity of the background noise increases relative to the vocalized word) if the subject correctly identifies the earlier presented vocalized word.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A system for designing hearing test protocols, the system comprising:
a computer system comprising a visual display; and
computer executable instructions executable to:
present on the display a plurality of user-selectable sound types;
register a sound type selected by the user from the plurality of displayed selectable sound types;
present on the display a plurality of selectable subject response types for the registered sound type;
register in association with the registered sound type a subject response type selected by the user from the plurality of selectable subject response types; and
create a computer-executable and portable hearing test protocol for testing a subject's hearing, the hearing test protocol comprising:
digital code encoding a test sound, based on the registered sound type and processable to make the test sound audible to the subject; and
computer executable instructions, based on the registered subject response type and executable to generate a computer-based response interface for registering the subject's response to the test sound.

2. The system according to claim 1, wherein the computer system is operable to:
present, based on the selected sound type, a sound configuration interface for configuring one or more properties of the test sound; and
encode the test sound in the test protocol based on the one or more test sound properties configured through the sound configuration interface.

3. The system according to claim 1, the computer system being operable to:
present a plurality of selectable subject response types based on the selected sound type.

4. The system according to claim 2, wherein the sound configuration interface is operable to configure the hearing test sound to comprise a sequence of multiple test sound presentations, and register a selection of a sequence rule for changing at least one property of the test sound during each presentation in the sequence.

5. (canceled)

6. The system according to claim 4, wherein the sequence rule is defined such that the at least one property of the test sound presentation later in the sequence is responsive to a subject response to a test sound presentation earlier in the sequence, as registered by the response procedure.

7. The system according to claim 1, wherein the hearing test protocol comprises computer executable instructions executable to direct collection and storage of subject responses for subsequent analysis, and to perform an analysis of the collected subject responses to assess the subject's hearing, a format of the analysis being defined and associated with the registered response type by the user.

8-9. (canceled)

10. The system according to claim 7, wherein the computer system is operable to adjust the one or more properties of the test sound encoded in the hearing test protocol responsive to a result of the analysis of the collected subject responses.

11. The system according to claim 7, wherein the computer executable instructions comprised in the computer system is executable to associate, within a database, the created hearing test protocol with subject responses collected from a plurality of executions of the created hearing test protocol performed by different subjects of a population of subjects.

12. The system according to claim 11, wherein the computer executable instructions comprised in the computer system is executable to provide statistics for analyzing the subject responses collected from the population of subjects for the created hearing test protocol.

12. (canceled)

13. The system according to claim 1, wherein:
the visual display provides a user with a first plurality of icons, each icon representing a type of sound, which icons the user manipulates to select the desired sound type of the test sound for transmission to the subject; and/or
the visual display provides the user with a second plurality of icons, each icon representing a subject response type for registering a subject response to said test sound, which second plurality of icons the user manipulates to select the desired subject response type.

14-39. (canceled)

40. A computer-based method of defining a computer executable hearing test protocol, the method comprising:
presenting on a display a plurality of user-selectable sound types;
registering a sound type selected by the user from the plurality of displayed selectable sound types;
presenting on the display a plurality of selectable subject response types for the registered sound type;
registering in association with the registered sound type a subject response type selected by the user from the plurality of selectable subject response types; and
creating a computer-executable and portable hearing test protocol for testing a subject's hearing, the hearing test protocol comprising:
digital code encoding a test sound, based on the registered sound type and processable to make the test sound audible to the subject; and
computer executable instructions, based on the registered subject response type and executable to generate a computer-based response interface for registering the subject's response to the test sound.

41. The method according to claim 40, further comprising:
presenting, based on the selected sound type, a sound configuration interface for configuring one or more properties of the test sound; and
encoding the test sound in the test protocol based on the one or more test sound properties configured through the sound configuration interface.

42. The method according to claim 40, further comprising:
presenting a plurality of selectable subject response types based on the selected sound type.

43. The method according to claim 41, wherein the sound configuration interface is operable to configure the test sound to comprise a sequence of multiple test sound presentations, and to register a selection of a sequence rule, defined by the user, for changing at least one property of the test sound during each presentation in the sequence.

44. (canceled)

45. The method according to claim 43, wherein the sequence rule is defined such that the at least one property of the test sound presentation later in the sequence is responsive to a subject response to a test sound presentation earlier in the sequence, as registered by the response procedure.

46. The method according to claim 40, wherein the hearing test protocol comprises computer executable instructions executable to direct collection and storage of subject responses for subsequent analysis, and to perform an analysis of the collected subject responses to assess the subject's hearing, a format of the analysis being defined and associated with the registered response type by the user.

47-48. (canceled)

49. The method according to claim 46, wherein the computer system is operable to adjust the one or more properties of the test sound encoded in the hearing test protocol responsive to a result of the analysis.

50. The method according to claim 46, further comprising associating, within a database, the created hearing test protocol with subject responses collected from a plurality of executions of the created hearing test protocol performed by different subjects of a population of subjects.

51. The method according to claim 50, further comprising analyzing the subject responses collected from the population of subjects for the created hearing test protocol.

52. The method according to claim 40, further comprising:
accessing an instruction set to generate the visual display comprising a first plurality of icons, each icon representing a type of sound defining a test sound for transmitting to a subject; and
registering a manipulation of at least one icon from the first plurality of icons representing a desired sound type, for selecting the desired sound type to define the test sound.

53. The method according to claim 40 further comprising:
accessing the instruction set to generate a second plurality of icons in the visual display, each icon representing a subject response type;
registering a manipulation of at least one icon from the second plurality of icons representing a desired subject response type.

54-68. (canceled)
